(19) United States
(12) Patent Application Publication
(10) Pub. No.: US 2010/0168634 A1
(43) Pub. Date: Jul. 1, 2010

(54) MULTI DIRECTIONAL STRETCH TUBULAR BANDAGES

(75) Inventors: Raymond Charles Leeming, Oldham (UK); Damian Antonio Fernandez, Oldham (UK)

Correspondence Address: DUANE MORRIS LLP - Philadelphia;IP DEPARTMENT, 30 SOUTH 17TH STREET, PHILADELPHIA, PA, 19103-4196, US

(73) Assignee: MEDLOCK MEDICAL LIMITED, Oldham (UK)

(21) Appl. No.: 12/513,345

(22) PCT Filed: Oct. 24, 2007
(86) PCT No.: PCT/GB2007/004043

§ 371 Date: Feb. 1, 2010

(30) Foreign Application Priority Data
Nov. 3, 2006 (GB) 0621926.5

(51) Int. Cl. A61F 13/00; A61L 15/46; A61F 15/00

(52) U.S. Cl. 602/44; 602/48; 602/76

(57) ABSTRACT

A stretchable bandage comprises a knitted fabric comprising a plurality of yarns (b), (c) of a first inelastic material, e.g. viscose knitted together with yarns (a}, (d), of a second elastically extensible material (e.g. elastane). As shown, the yarns may be knitted from a pair of yarns, one viscose (c) and one elastane (d) fed together and occupying a common yarn path, and separated from the next such pair of yarns by a viscose yarn (b) and an elastane yarn (a) fed separately as single yarns.

This invention relates to improvements in stretchable knitted tubular bandages for use in the applications support, retention, undercast and compression. These bandages are well known and are used in the treatment of a variety of conditions, usually affecting the arms or legs of a patient, such as injuries, disease or other uses requiring a gentle but firm inward pressure. Retention bandages may be specially for securing primary dressings on wounds or ulcers. It could be used as lightweight bandage for the treatment of atopic eczema using the Wet Wrapping, Dry Wrapping or Patch Wrapping treatment therapy. Stretchable knit tubular bandages may be used as an undercast stockinette.

Before 1960, crepe bandages were widely used as the creped fabric has some elasticity, and if the bandage is applied in a stretched state, is effective in providing some inward pressure. In GB840523 is described a tubular bandage which exerts an inward pressure at all points and conforms to the shape of the part of the torso or limbs to which it is applied, comprising a parallel seamless bandage made in a continuous length and knitted so as to possess a degree of circumferential longitudinal stretch and which includes one or more circumferentially arranged evenly pitched elastic or resilient elements such as rubber filament. The elastic or resilient element is inert or limp in the non-tensioned stated of the bandage, but becomes tensioned as the bandage is extended, and when applied about a body part effects tension such as to make the bandage conform closely to the part and exert a minimal inward pressure for retention with a higher level for support at all points.

GBA2167459 describes a variant of the tubular bandage described in the above document wherein the spacing or pitch of the elastic element is variable by varying the knit density of the stocking knit so that the elastic yarns can move more closely together in the lower density regions. This enables a greater density of elastic yarn to be provided in predetermined parts of the bandage to enable increased pressure to be applied where required without folding over the bandage to provide two or more layers.

These bandage constructions have been used successfully and are sold under the Trade Marks TUBIFAST and TUBIGRIP by Medlock Medical Limited, successors to the original patentees Seton Products Ltd. However whilst a good degree of elasticity, and grip is provided laterally of the bandage, which is provided by elastic thread inlays, the elasticity in the longitudinal direction is limited to the degree of elasticity inherent in the yarns, which is considerably less than that available circumferentially from the knit loop structure.

For use on straight parts of limbs, the lateral stretch is often adequate to retain dressings or topical preparations in place. However, at joints such as elbows or knees, movement of the joint caused by flexing of the limb may allow the bandage and or primary dressing applied to the limb at or close to the joint to become creased or displaced.

It is an object of the invention to provide a bandage which is capable of being extended under elastic tension in both longitudinal and lateral directions.

A stretchable bandage according to the invention consist of a knitted fabric comprising a plurality of yarns of a first substantially inelastic material, which are knitted together so that loops of said yarns are interconnected, characterised in that yarns of a second elastically extensible resilient material are interlooped with the yarns of said inelastic material.

The yarns of resilient material are preferably interlooped with the yarns of inelastic material in such a way that the yarns of resilient material interlooped with each of respective pairs of inelastic yarns in the respective inter knitting of the inelastic yarns. Elastic yarns may be so introduced at each second or third pair of interknitting inelastic yarns.

In a preferred embodiment, the elastic yarns may comprise elastane yarns, and the relatively inelastic yarns may comprise viscose yarns. Of course, yarns of any other suitable material may be used, including natural fibres such as cotton, manmade fibres such as nylon and other elastic materials may be used for the resilient elastic yarns that could be latex or non latex based. Both inelastic and elastic yarns may contain antimicrobial properties such as metals, alginates, polysaccharides, chitosan and/or other chemical treatments.

The bandage according to the invention may be a tubular bandage, manufactured on a circular knitting machine. The latter may be modified to provide extra yarns feeds to introduce the resilient yarns to the knitting machine. It is not excluded that the bandage according to the invention comprises a flat bandage, knitted on a warp knitting machine, to form an elongate strip for use in application as a wrap around or helical bandage. Nor may it exclude a bandage knit on a flat knit machine to provide a flat or tubular bandage. The bandage may be finished with an antimicrobial treatment.

The bandage of the invention has been found to be capable of resilient stretching and recovery in both the longitudinal and lateral directions, in contrast to the known comparable treatment bandages which are only stretchable to any significant extent in the lateral direction.

In a modified of the invention, the elastic yarns may be used in a conventional stockinette, plain knit or double jersey knit in place of a non-elastic yarn, say every third or fourth yarn, the other yarns being non-elastic such as viscose.

A preferred embodiment of the invention will now be described with reference to the accompanying drawings wherein:

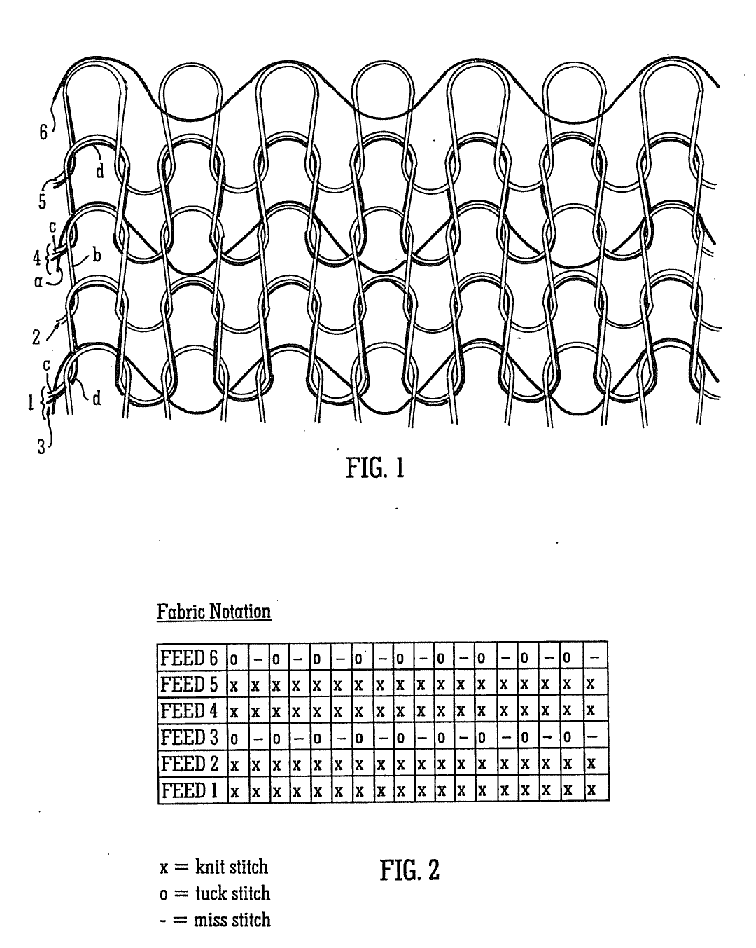

FIG. 1 is a diagrammatic representation, on an enlarged scale of the knit structure of a bandage according to the invention; and

FIG. 2 is a fabric notation tabulation showing the knit structure of the knitted fabric of FIG. 1.

In the representation of the example of a knitted fabric in a bandage according to the present invention, there is shown the loop patterns and yarn paths of a stockinette knitted fabric, knitted as a tube in a six feed circular knitting machine. In the drawing, inelastic viscose yarns are shown white, whilst elastic, stretchable and relaxable elastane yarns are shown in black, to highlight the latter.

The diagram of FIG. 1 shows 6 yarn feeds, numbered 1 to 6 from bottom to top. Feeds 1 and 4 are arranged to each feed a pair of yarns, namely a viscose yarn c, and an elastane yarn d. Feeds 2 and 5 each feed a single viscose yarn b, and feeds 3 and 6 each feed a single elastane yarn. The yarns c and d (pair of elastane and viscose yarn) follow the same knit path and are fully incorporated into the knit structure, the single viscose yarns b also follow a conventional yarn path in the knit, but the single elastane yarns a are effectively looped in tuck stitches about alternate stitch rows. The yarns a provide elasticity in the circumferential direction, whilst the elastane yarns d fully incorporated into the knit, provide substantially enhanced elasticity in the longitudinal direction.

From the stitch notation table in FIG. 2 it will be seen that the viscose yarns from feeds 1 and 2, and feeds 4 and 5 are formed into conventional knitted stitches. It should be noted that an elastane is knitted with the viscose on every second knitted course. This ratio may be altered to suit the required end use of the bandage. To say, that the knitted elastane yarn could be introduced into the knit to be interlooped with the non-elastic yarn, be it viscose or other suitable yarn on every third, fourth or fifth knitted course.

This arrangement enables the knitted fabric to have longitudinal elasticity, as well as the lateral elastically which was provided in the prior art tubular bandages. In the latter, elastic yarns, originally rubber, more recently elastane, were simply extended along the stitch rows of the knitted fabric, with alternate stitches over and under the elastic yarns.

This enabled only stretching along the elastic yarn; the only elasticity lengthwise was due to the limited inherent stretch of the knitted fabric itself and no more than for unelasticated knits.

The knitted fabric as described is preferably knitted in tubular form, and maybe made up into tubular bandages. These may be used as retention bandages to secure primary dressings, or support bandages, to apply pressure to limb or other parts, and for use with emollients or other topical applications for treatment of skin conditions such as eczema or psoriasis.

The tubular bandage thus provided, is of particular value for retaining primary dressings on or close to joints such as elbows or knees, where flexure of the joint and or muscle movement has tended to displace or crease or otherwise compromise a dressing, since the longitudinal elasticity now provided for enables the movement to the accommodated, which was not possible when only radial elasticity was provided. Folds in bandages can create pressure points and this invention will reduce the incidence of it. This also enables an improvement in patient comfort and further aids patient compliance.

The fabric may also be used in strip bandages for application by winding onto a site of use. This may be formed by cutting from a tubular knitted fabric, or knitted on a flat bed or warp knitting machine. This is feasible because being tucked into the knit, anchoring of the elastane yarns is not the problem it would be with helical inlaid elastic yarns.

It will be seen that in addition to a knitted fabric, the invention also provides a tubular bandage made from the fabric, a flat strip bandage made from the fabric, and any other form of topical dressing or treatment system which can be made using the fabric according to the invention.

1. A stretchable bandage consisting of a knitted fabric comprising a plurality of yarns of a first substantially inelastic material, which are knitted together so that the loops of said yarns are interconnected, characterised in that the yarns of a second elastically extensible resilient material are interlooped with the yarns of said inelastic material whereby the bandage is stretchable both radially and longitudinally.

2. A stretchable bandage according to claim 1, further characterised in that the yarns of resilient material are interlooped with the yarns of inelastic material in such a way that the yarns of resilient material are interlooped with each of respective pairs of inelastic yarns in the respective inter knitting of the inelastic yarns.

3. A bandage according to claim 1 wherein the elastic yarns comprise elastane yarns, and the relatively inelastic yarns comprises viscose yarns.

4. A bandage according to claim 1 wherein the elastic and the inelastic yams contain anti-microbial additives.

5. A bandage according to claim 1 wherein the bandage is a tubular bandage.

6. A bandage according to claim 1 wherein the bandage comprises a flat bandage in the form of an elongated strip for use in application as a wrap around or helical bandage.

7. A bandage according to claim 1 wherein the knitted fabric comprises pairs of yarns comprising an inelastic yarn and an elastic yarn fed together in a common yarn path, and connected by further elastic and inelastic yarns fed singly into the knit.
